(19) United States
(12) Patent Application Publication
(10) Pub. No.: US 2012/0264689 A1
(43) Pub. Date: Oct. 18, 2012

(54) METHODS AND COMPOSITIONS FOR SKIN REGENERATION

(75) Inventor: Nancy K Mize, Mountain View, CA (US)

(73) Assignee: Genogen, Inc., Mountain View, CA (US)

(21) Appl. No.: 13/501,098

(22) PCT Filed: Oct. 7, 2010
(86) PCT No.: PCT/US10/51830

§ 371 Date: Jun. 25, 2012

Related U.S. Application Data
(60) Provisional application No. 61/380,671, filed on Sep. 7, 2010.
(60) Provisional application No. 61/278,576, filed on Oct. 7, 2009.

(51) Int. Cl. A61K 38/18; A61P 17/02; A61K 8/64; A61Q 7/00; A61P 27/02; A61P 31/18; A61P 3/10; A61P 17/00; A61Q 19/08; B82Y 5/00

(52) U.S. Cl. 514/8.6; 514/7.6; 977/773; 977/906; 977/788

(57) ABSTRACT

Compositions and methods for inducing tissue differentiation and growth within a localized area of application in humans or other animals are provided. Compositions comprising a biological or pharmacologically active agent such as Insulin-like Growth Factor-1 (IGF-1) and sustained-release nanoparticle formulations comprising IGF-1 and a matrix forming component such as hyaluronan for use in skin repair and regeneration are provided. Nanoparticles of the invention activate endogenous stem cells in the local area of administration to produce new dermal cells and tissues.

### CROSS-REFERENCE TO RELATED APPLICATIONS

This patent application claims priority of U.S. Provisional Patent Application Ser. No. 61/278,576 filed Oct. 7, 2009 entitled “Methods and composition for cell, tissue, and organ regeneration and repair,” and U.S. Provisional Patent Application Ser. No. 61/380,671 filed Sep. 7, 2010 entitled “Methods and composition for skin regeneration,” which are incorporated herein in their entirety by reference.

### TECHNICAL FIELD OF THE INVENTION

The present invention relates to compositions and methods for inducing tissue and cell regeneration by differentiation and growth within a localized area of application in humans or other animals. Specifically, the invention relates to compositions comprising a biological or pharmacologically active agent such as Insulin-like Growth Factor-1 (IGF-1). More specifically, the invention relates to sustained-release nanoparticles comprising IGF-1 and a matrix forming component such as hyaluronan for use in skin repair and regeneration.

### BACKGROUND OF THE INVENTION

According to a recent report, Worldwide Markets and Emerging Technologies for Tissue Engineering and Regenerative Medicine, distributed by Life Science Intelligence (LSI), the largely untapped global market potential for tissue engineering and regenerative medicine products will exceed $118 billion by 2013. (www.medicalnewstoday.com/articles/141341.php; accessed Aug. 16, 2010).

Skin has an intrinsic capacity to repair itself unaided. Therefore, in the case of a simple external injury, the skin can be regenerated by its self-repair function. However, in the case of refractory wounds, such as serious burns, and complex wounds from radiation exposure, it is difficult to completely regenerate the skin. The global market for advanced wound care products which was worth $4.6 billion in 2007, is expected to reach $12.5 billion by 2012. (Wound Care Markets, 4th. Edition, Vol. I: Skin Ulcers. Kalorama Information. 2009).

Skin aging is a complex biological process affecting various layers of the skin and the hypodermis, but whose major effects are seen in the dermis. There are two biologically independent aging processes that occur simultaneously. The first is intrinsic aging, which affects skin as well as, most likely, the internal organs. The second is extrinsic aging or photo-aging which is the result of exposure to the elements, primarily ultraviolet irradiation. Thus, the most noticeable changes on facial and neck skin, the primary areas that patients are concerned about, result from the combination of intrinsic and extrinsic aging processes.

The worldwide market for dermal fillers is projected to continue to grow at double digit or near double-digit rates, approaching $950 million by 2010. (Dermal Filler Market: More than Skin Deep. Mary Thompson, Scott Huntley, Medtech Insight Mar. 1, 2008).

Both antiretroviral therapies and the human immunodeficiency virus (HIV) itself cause HIV-associated lipodystrophy characterized by body composition changes. The body composition changes include two independent conditions: (a) excess fat accumulation, where deep abdominal fat (visceral adipose tissue or VAT) surrounds the internal organs—lipohypertrophy; and (b) fat loss where subcutaneous fat loss typically occurs in the face, limbs and buttocks—lipoatrophy.

By 2012 approximately 380,000 patients treated with antiretrovirals will have HIV-associated lipodystrophy in North America and Europe and that the potential total market size for this condition will be between US$811 million to US $1.3 billion. (www.theratech.com/en/products-therapeutic-peptides/lipodystrophy.php; accessed Aug. 16, 2010)

These conditions, skin aging, complex wounds and burns, and lipodystrophy, result from a disruption of the hypodermis, which is predominantly populated with adipocytes (fat cells) and hair follicles, both sources of stem cells.

The process of wound healing includes the following steps: (1) recognition of the damaged area by the inflammatory cells, and subsequently by the connective tissue cells and epidermal cells; (2) shrinkage of the wound area; and (3) granulation and re-epithelialization. The cells, various factors, cytokines and secretions involved in each stage of this process have been largely identified.

The cellular regeneration of the skin is maintained by different adult stem/progenitor cell subpopulations localized within the specialized microenvironments, niches in interfollicular epidermis (IFE), sebaceous gland and hair follicle bulge region (Mimeault, M. and Batra, S. K. (2010), Recent advances on skin-resident stem/progenitor cell functions in skin regeneration, aging and cancers and novel anti-aging and cancer therapies. Journal of Cellular and Molecular Medicine, 14: 116-134).

Small clusters of undifferentiated and unipotent keratinocyte stem cells (KSCs) with stem cell-like properties and expressing cytokeratins have been identified near basement membrane in the basal layer of epithelial compartment. KSCs provide critical roles in participating to the continuous replenishment of mature keratinocytes of epidermis in homeostatic conditions and after skin injuries. A complex signaling network including insulin-like growth factor (IGF-1; also known as Somatomedin C) is involved in the proliferation and/or differentiation into keratinocytes during skin tissue regeneration. (Fuchs E. Skin stem cells: rising to the surface. J Cell Biol. 2008; 180: 273-84.)

The study of artificial skin reparation began with just modifying materials which cover the wound for temporary protection of the wound surface, and now, has reached to induction of more aggressive treatments by using agents such as cytokines and growth factors that are related to wound healing. However, there is a need for a technique capable of facilitating and inducing epithelialization that is useful for regeneration and repair of various degrees of skin wounds, from simple to refractory, including regeneration of the hypodermis.

Use of a skin substitute for the treatment in patients who have complete loss of full-thickness skin has been available. A skin substitute incorporating epidermal cells is called cultured epidermis, a skin substitute incorporating dermis fibroblasts is called cultured dermis, and a skin substitute incorporating both of them is called cultured skin.

Currently available as cultured dermis are products having different matrixes into which fibroblasts are incorporated, such as TransCyte® and Dermagraft®. However, cultured dermis, as well as cultured epidermis, do not have an ability to induce epithelialization in large wounds. Cultured skin incorporating epidermal cells and fibroblasts are available as Apligrag® (NOVARTIS Pharma) and VivoDerm® (Bristol-Myers Squibb). However, there are problems regarding the affinity between cultured epidermal layer and dermal layer, and insufficiency in clinical effect obtainable. In addition, there is a concern for the safety of the current skin substitutes that are highly dependent on animal collagen or human plasma components.

Donor tissue is extremely scarce, and treatment of transplant recipients with immunosuppressant drugs creates substantial health risks for the transplant recipient. Transplantation of autologous or allogenic tissue has been used, but with limited success.

An alternative strategy is the use of implanted stem cells to promote tissue growth in vivo or to generate cultured tissues for transplantation (reviewed by Vogel, Science 283:1432-1434 (1999)). Stem cells are cells that are capable of self-renewal and give rise to cells of more specialized function (reviewed by Blau, Cell 105:829-841 (2001); Weissman, Cell 100:157-168 (2000)). Methods for skin regeneration using mesenchymal stem cells comprising a multilayer skin equivalent having (i) a scaffold layer incorporated with dermis-forming cells, and (ii) a keratinocyte layer is disclosed in EP0953040. However, in addition to ethical concerns the use of extrinsic stem cells raise concerns regarding tumorigenicity caused by undifferentiated pluripotent cells as well as immunogenicity caused by allogenicity.

Therefore, there is a need for a product useful for regeneration and repair of skin by activating endogenous (pre-existing) stem cells thus avoiding the many drawbacks involved in current stem cell therapies.

### SUMMARY OF THE INVENTION

Reduction in stem cell activity with aging plays an important role in slowing healing and tissue regeneration. The present invention is based on the discovery that adult skin cells can be induced in situ to undergo differentiation into the many cell types required to regenerate tissues.

Avoiding the drawbacks associated with conventional stem cell therapy procedures such as stem cell isolation, preparation, surgery, extraction, etc. the present invention provides methods and compositions for endogenous stem cell activation by localized delivery of stem cell activators and growth matrix.

This invention provides compositions and methods for sustained release of growth factors at localized sites for activating endogenous stem cells for a period of time sufficient for repair and regeneration of endogenous skin cells and tissues. The method takes advantage of nature's own method of repairing tissues by augmenting such processes.

As described in further detail infra the present invention relates to a product that induces differentiation and growth within the localized area of application of the product into humans or other animals. The product is useful for regeneration and repair of aging, lesions, wounds, diseases, or abnormalities, particularly for regenerating and/or repairing skin. A method of application of the product for regeneration and repair of skin is described.

This product may be used for regeneration of skin by topical application, injection, or application of a patch to the affected area. An example method is provided for injection in the treatment and repair of defects of skin, as in wound healing and cosmetic applications. To stimulate the production of new skin tissue, the dermis or other suitable area is injected with a needle and syringe containing a biological or pharmacologically active agent such as Insulin-like Growth Factor-1 (IGF-1), with or without additional substance(s) such as collagen, hyaluronan (also called hyaluronic acid or hyaluronate), and/or a bioabsorbable gel and/or a stent or insert. The additional substance(s) serve as a carrier, as a matrix for growth, as an aid in mechanical stimulation of stem cells, and/or to release the bioactive agent in a controlled manner. Hyaluronin, collagen, or bioabsorbable gel, with its high biocompatibility, may serve as biomaterial scaffold for cell and matrix growth induced by this method. The stent or insert may also serve as a permanent or temporary scaffold or matrix for growth.

The product may be injected into the dermis in the doctor's office for example, with repeated injections over time in a similar manner as hyaluronan is now injected in the physician's office. The method is also useful in numerous applications including other tissue repair.

These compositions and methods are useful for treatment and therapy of aging, lesions, wounds, diseases, or abnormalities, particularly for regenerating and/or repairing skin.

The methods of treatment encompass both prophylactic and therapeutic applications.

In one aspect of the invention, the formulation and method of administration of IGF-1 disclosed herein reduces side-effects and adverse reactions, such as hypoglycemia, typically associated with current methods of administration of IGF-1 which results in high serum concentrations over prolonged periods of time. Surprisingly, the inventor of the present invention has found that providing localized delivery of a single dose of IGF-1 at 20, 50, 100, 200, 300, 200, or 500 ng (approximately 10,000× lower than the currently prescribed dose of IGF-1 for children) combined with HA (or other bioabsorbable polymer, microneedles, stent or insert) for sustained release, and administered locally over a small area (0.1 to 10 cm², preferably 0.25 to 6 cm², surface area, or 2 to 1000 cm³ infused volume), produces an unexpected result of tissue regeneration in the local area of injection. Further, the serum concentration of IGF-1 administered in this manner remains in the normal physiological range, thus avoiding undesirable side-effects and adverse reactions.

These and other aspects will become apparent from the following description of the preferred embodiment taken in conjunction with the following drawings, although variations and modifications therein may be affected without departing from the spirit and scope of the novel concepts of the disclosure.

### BRIEF DESCRIPTION OF THE DRAWINGS

The following drawings form part of the present specification and are included to further demonstrate certain aspects of the present disclosure, the inventions of which can be better understood by reference to one or more of these drawings in combination with the detailed description of specific embodiments presented herein. The patent or application file contains at least one drawing executed in color. Copies of this patent or patent application publication with color drawing(s) will be provided by the Office upon request and payment of the necessary fee.

FIGS. 1A-1C shows the effects at 14 days after injection of PBS (controls; FIG. 1A), HA (damaged; FIG. 1B), or GG002, a bioactive agent IGF-1 with hyaluronan (regenerated; FIG. 1C), in a syringe injected into a local area of the dermis of mice. Each panel shows 4 different mice samples, 6 mm punch biopsy sliced and stained with Hematoxylin and Eosin (H&E) and viewed at 5× magnification.

### DETAILED DESCRIPTION OF THE INVENTION

The terms used in this specification generally have their ordinary meanings in the art, within the context of the invention, and in the specific context where each term is used. Certain terms that are used to describe the invention are discussed below, or elsewhere in the specification, to provide additional guidance to the practitioner regarding the description of the invention. For convenience, certain terms may be highlighted, for example using italics and/or quotation marks. The use of highlighting has no influence on the scope and meaning of a term; the scope and meaning of a term is the same, in the same context, whether or not it is highlighted. It will be appreciated that same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and in no way limits the scope and meaning of the invention or of any exemplified term. Likewise, the invention is not limited to various embodiments given in this specification.

Unless otherwise defined, all technical and scientific terms used herein have the same meaning as commonly understood by one of ordinary skill in the art to which this invention pertains. In the case of conflict, the present document, including definitions will control.

The present invention is based on promoting, stimulating or inducing cell migration and/or proliferation, which may have use in wound healing, tissue engineering, cosmetic and therapeutic treatments such as skin replacement and skin replenishment and treatment of burns where epithelial cell migration and proliferation is required.

No exogenous stem cells need be obtained, extracted, isolated, or purified; procedure can be performed in the doctor's office; the bioactive agents and other components can be bioabsorbable; the drug delivery is local at the site of injury, e.g. local skin delivery; the procedure is minimally invasive.

In both mice and humans, a layer of cells at the base of the skin contains stem cells that can develop into the specialized cells in the layers above. Two transcription factors C/EBPα and C/EBPβ are co-expressed in basal keratinocytes, and are coordinately up-regulated as keratinocytes undergo terminal differentiation (Lopez R G et al., Nat Cell Biol. 2009 11(10):1181-1190.)

Using an animal model, it was found that stem cells in the hair follicle can be enlisted to help heal wounds in the skin. (Ito, M., et al. Nat Med. 2005; 11(12):1351-1354.) This finding, suggested a therapeutic target for the development of drugs to encourage and promote wound healing. Studies on stem cells in the adult hair follicle (HF) have uncovered a veritable menagerie of exceptionally diverse and dynamic keratinocytes with stem cell properties located in distinct regions of the HF. Although endowed with specific functions during normal hair follicle maintenance, the majority of these cells can act as multipotent stem cells. (Jaks, V., et al., Exp Cell Res. 2010; 316(8):1422-1428).

A complex network of signaling cascades such as EGFR, Notch, insulin-like growth factor (IGF-1)/IGF-R1, immunoglobulin-like domains 1 (Lrig1), Myc, transforming growth factor-β (TGF-β) and Polycomb-group protein BMI-1 are involved in the stringent regulation of keratinocyte stem cells' (KSCs) proliferation and/or differentiation into keratinocytes during skin tissue regeneration. (Mimeault, M. and Batra, S. K. (2010). J. Cell. and Mol. Med., 14: 116-134).

In one embodiment, the bioactive agent delivered to the site for intended repair or regeneration of skin is IGF-1. After injection into the affected area, the bioactive substance (e.g. IGF-1) activates stem cells endogenous to the area to produce new tissue, e.g. dermal and subdermal cells and tissues.

The formulation for use in humans or other animals for the purposes of regeneration is comprised of a bioactive agent such as Insulin-like Growth Factor-1 (IGF-1), alone or combined with either a bioabsorbable polymer such as Lactel®, collagen, hydroxyapatite, poly L lactic acid, elastin, microspheres, depleted human skin matrix, and/or hyaluronan or other suitable substance.

The IGF-1 used can be any pharmaceutically effective commercially available version approved by the FDA. In one embodiment, Increlex® (Tercica, Inc.), a recombinantly produced version identical to the natural hormone IGF-1, is used. Increlex® is the only isolated IGF-1 replacement therapy indicated specifically for the long-term treatment of growth failure in children with severe primary IGF-1 deficiency. Currently, the only FDA-approved treatment for using IGF-1 is Increlex®.

However, the current standard regimen for administration of IGF-1 results in high serum concentrations of IGF-1 for prolonged periods of time, resulting in adverse reactions and undesirable side-effects. The standard treatment dosage administered to IGF-1 deficient children as Increlex® is 0.06 to 0.12 mg/kg 2× day, or 1.2 to 1.4 mg per day for a 20 kg child as administered twice daily for several years. (Increlex® package insert; www.increlex.com/hcp-full-prescribing-information.asp; accessed Aug. 30, 2010). According to the package insert, Increlex® results in a plasma concentration peak of IGF-1 in 3 hours in IGF-1 deficient children. Administration of Increlex® maintains an elevated plasma concentration in IGF-1-deficient children over 12 hours, with repeat dosing every 12 hours in order to achieve statural growth. As listed in the Increlex® package insert, the major adverse reaction (observed in over 40% of patients) is severe hypoglycemia including hypoglycemic seizures and loss of consciousness due to the high systemic concentration of IGF-1.

In contrast, the formulation and methods of administration of IGF-1 according to the current invention results in no measurable increase in existing plasma concentration of IGF-1 in 1, 2, 3, 4, 6, 8, 24, or 48 hours after delivery, and hypoglycemia is not observed.

As disclosed herein, for local delivery of the drug, the dose is at or near human physiological levels of IGF-1, and thus avoids side effects. The normal serum insulin-like growth factor-I (IGF-I) level is approximately 250 ng/mL. The dose response needed is between 20 ng/mL and 500 ng/mL for IGF-1. This dosage range is at least about 10,000× less than the systemic dose for Increlex® (500 ng/mL vs 10 mg/mL Increlex®) thus avoiding adverse reactions.

In addition to the approximately 10,000× less concentration of IGF-1, the present composition is delivered in a single dose, and the sustained release from that administration is below 500, 400, 300, 200, 100, or 50 ng/mL over 1, 2, 3, 4, 12, or 24 hours, respectively.

The composition further comprises a bio-absorbable/degradable matrix, forming a sustained release formulation comprising nanoparticles. The matrix comprising any of a bioabsorbable gel, collagen, hydroxyapatite, poly L lactic acid, elastin, microspheres, depleted human skin matrix, hyaluronan, insert, or stent may mechanically activate endogeneous stem cells and also act as a matrix or scaffold for cell growth.

In one embodiment the matrix-forming component comprises hyaluronan. Hyaluronic acid (“HA”) is a polysaccharide composed of D-glucuronic acid and N-acetyl-D-glucosamine. It has been used for transdermal drug delivery. HA is found on mammalian cell surfaces, in the basic extra cellular substances of the connective tissues of vertebrates, synovial fluid of joints, vitreous of the eye, tissue of human umbilical cord and in cocks' comb. It is the main component of the extracellular matrix. HA plays an important role in the mechanical support of the cells of many tissues, such as the skin, the tendons, the muscles and cartilage.

Nanoparticles from natural polymers, biocompatibles and biodegradables can be used for the controlled release of the active molecules they transport and their orientation towards the target tissues. Particles of hyaluronic acid in salt form, preferably the sodium salt of the polymers or hybridized with magnetic iron particles, with a diameter less than 180 nm, that incorporate an active ingredient, independent of its hydrophilic or hydrophobic nature are known. Procedures to produce such nanoparticles useful in methods of transdermal drug delivery using hyaluronic acid nanoparticles, are described in U.S. Pat. No. 7,371,738 (Mohapatra et al.)

Like other nanoparticles, HA has several advantages as a carrier of genes, drugs or proteins to these cells. It is less immunogenic or non-immunogenic. Its molecular structure is common in all mammals (HA is a major component of the extracellular matrix of all tissues). Derivatives of HA have unique properties for specific biomedical applications without any known adverse effects.

The amount of hyaluronic acid in compositions according to the invention can be 2, 4, 6, 8, 10, 20, 30, 40, 50, or 60 mg/mL. In preferred embodiments the concentration ranges between 4 mg/mL and 60 mg/mL of Hyaluronic Acid.

Alternatively, the matrix, e.g. hyaluronic acid, which provides the scaffold, may be impregnated, coated or linked to IGF-1. IGF-1 may be imbedded/suspended/coated in the hyaluronic acid.

A treatment method for restoring of age related tissue loss in the face or selected areas of the body is disclosed which includes injecting an injectable composition containing a growth factor and hyaluronic acid as a carrier into the dermis, the hypodermis, or both, in various areas of the face, or selected areas of the body of a person to stimulate collagen, elastin, or fat cell production, thereby restoring age related tissue loss in the face and selected areas of the body is disclosed in U.S. Patent App. Ser. No. 20060073178 (VC Giampapa)

A “hydrogel” is a substance formed when an organic polymer, which can be natural or synthetic, is set or solidified to create a three-dimensional open-lattice structure that entraps molecules of water or other solutions to form a gel. Solidification can occur by aggregation, coagulation, hydrophobic interactions, cross-linking, or similar means. Preferably, the hydrogels used in conjunction with in situ stem cells or spore-like cells and their progeny form a matrix that the cells are retained at the application site, and other cells subsequently migrate into the matrix. This matrix-cell combination enhances new cell growth at the application site. The hydrogels are also biocompatible (e.g., they are not toxic to cells). The “hydrogel-cell composition” referred to herein is a suspension that includes a hydrogel and a stem cell or spore-like cell or its progeny within the area of application.

Hyaluronic acid hydrogels can be polymerized in situ, are biodegradable, and can serve as a tissue adhesive, a tissue separater, a drug delivery system, a matrix for cell cultures, and a temporary scaffold for tissue regeneration. (U.S. Pat. No. 6,630,457; Aeschlimann et al.)

One example of a commercially available hyaluronan suitable for the present invention is RESTYLANE® (Q-med, Seminariegatan, Uppsala).

Alternatively, other matrix forming components can also be incorporated in the formulation. Examples of these components are described below.

LACTEL® Absorbable Polymers are biodegradable polymers comprised of glycolide, lactide and ε-caprolactone monomers for use in medical applications. (Durect Corp. Cupertino Calif.).

Collagen, a major component of the extracellular matrix, is a fibrous protein that provides tensile strength to tissues. Medically, collagen has been widely used in such diverse applications as dermal augmentation, wound repair/surgical hemostasis, drug delivery, tissue engineering, and as coatings to increase the biocompatibility of many medical devices. Recombinantly produced human collagen (rhCIII) FG-5030® is available from Fibrogen, Inc. San Francisco Calif.)

Alternatives for the Hyaluronic Acid component are selected from: Hydroxyapatite; Collagen; Cross-linked Hyaluronic acid; multiple different MWs HA; Any suitable matrix forming agent; bioabsorbable gels; transplant-based fillers, e.g. fibroblasts or fat cells; synthetic Polymer-Based Fillers, e.g. polyacrylamide gel (Aquamid®), polyalkylimide gel (Bio-Alcamid®), Poly-L-lactic-acid (Sculptra/NewFill®), Polymethylmethacrylate (ArteFill/ArteColl®), hydroxyapatite, elastin, microspheres, depleted natural or synthetic human skin matrix.

In an important aspect of the invention, the local delivery of the composition creates a depot for sustained local release.

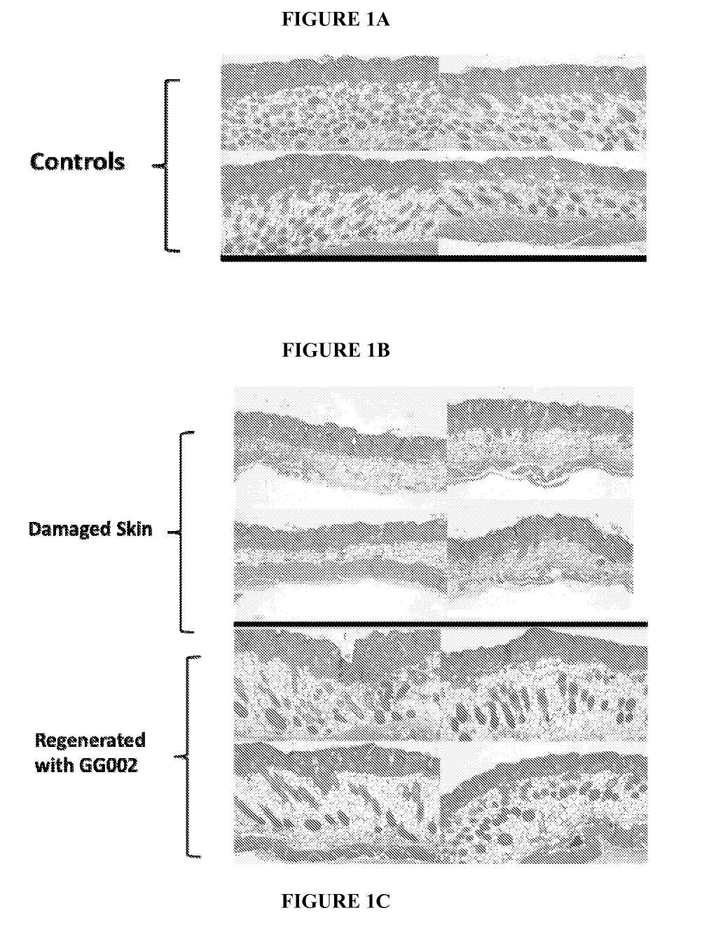

The sustained-release formulation of the present invention comprising a combination of IGF-1 and HA, when administered locally in low near-physiological levels, produces the surprising result of skin tissue regeneration. Whereas, according to the currently approved regimen for administration, IGF-1 (Increlex®) is delivered two times daily over several years, reaching thousands of doses (0.06 to 0.12 mg/kg 2× day, or 1.2 to 1.4 mg per day for a 20 kg child) in IGF-1-deficient children in order to increase statural growth, the inventor has found that a single dose of IGF-1 at 20, 50, 100, 200, 300, 200, or 500 ng per dose (approximately 10,000× lower than current IGF-1 dosage administered to children) combined with HA (or other bioabsorbable polymer, microneedles, stent or insert) for sustained release, and administered locally over a small area (0.1 to 10 cm², preferably 0.25 to 6 cm², surface area, or 2 to 1000 cm³ infused volume), gives the unexpected, surprising result of dermal tissue regeneration in the local area of injection (see FIGS. 1A-1C). Further, such formulations typically result in release of less than 500, 400, 300, 200, 100, or 50 ng/mL of IGF-1 over 1, 2, 3, 4, 12, or 24 hours, respectively, which is insufficient to cause side effects such as hyperglycemia.

The loss of soft tissue volume and position, unmasking underlying skeletal contours, is responsible for much of the aging appearance. Rejuvenation is achieved by restoring soft tissue volume and repositioning, hence the role of facial fillers.

The layer of subcutaneous fat immediately under the dermis, corresponding to the hypodermis is protective, mechanically and chemically. (Office-Based Cosmetic Procedures and Techniques, ed. Eremia S. Cambridge University Press, 2010.) Loss of volume of subcutaneous tissue, due to fat atrophy contributes significantly to the aging process.

This is remedied by administration of the formulations of the invention comprising IGF-1 by (a) injection into the mid-dermis or hypodermis (subcutaneous), (b) microneedle delivery, (c) topical creams, lotions, or sealant dressings (patches) and other suitable means for delivery.

Application of the formulation can be varied out by topical application, injection, or application of a patch to the affected area and/or insertion of a stent or insert or utilization of synthetic or natural skin. The procedure may be repeated to obtain the desired result. This product can be used in the doctor's office as hyaluronan injections are used in Restylane® (Medicis Aesthetics Inc., Scottsdale Ariz.), or during or after other medical treatments, or for regeneration or repair of organs, tissues, or cells, damaged by aging or injury or surgery or disease or other mechanisms.

In one embodiment, the composition is administered as multiple, partial dose injections into the mid to deep dermis, or hypodermis in a pattern of injection in order to distribute the composition in small doses over a local area, which according to the invention can be 0.25, 0.5, 0.75, 1.0, 2.0, 3.0, 4.0, 5.0, or 6.0 cm². This can also be accomplished by microneedles, patches, or topical crème using a diluted (up to 20,000×) IGF-1 formulation.

In several embodiments, the growth factor and the bioabsorbable polymer are applied simultaneously or sequentially at adjacent sites. This may be accomplished by injection of one component followed by injection of the second component within several minutes (typically less than 15 minutes, sometimes within less than 10 minutes or 5 minutes, preferably within two minutes) into the same injection site, or another site in near proximity (preferably within 2.0 cm or less, preferably within 0.5 cm).

Formulations according to the invention, with demonstrated therapeutic value, can be transported through the skin via unassisted or passive transdermal drug delivery. The main barrier to transport of molecules through the skin is the stratum corneum (the outermost layer of the skin). Devices including arrays of relatively small structures, sometimes referred to as microneedles or micro-pins, have been disclosed for use in connection with the delivery of therapeutic agents and other substances through the skin and other surfaces. The devices are typically pressed against the skin in an effort to pierce the stratum corneum such that the therapeutic agents and other substances can pass through that layer and into the tissues below. Alternatively, the needle-like projections may be created from the formulation with added hardeners that dissolve after penetration of the stratum corneum. Methods of manufacture and use of arrays of porous or hollow microneedles fabricated from metals, silicon, silicon dioxide, ceramic, and polymeric materials are known in the art. (See e.g., U.S. Pat. No. 6,503,231)

The methods of treatment may encompass both prophylactic and therapeutic applications.

Compositions and methods of the invention are suitable for several therapeutic indications including, but not limited to: fat pad regeneration; hair regeneration; skin regeneration with nerves & organelles; wound healing, including diabetic ulcers; burn healing; skin regeneration and repair; enhance skin quality or appearance; prevention or remediation of skin disorders; diminishment or abolishment of scar tissues; breast skin regeneration (after surgery); cosmetic applications, e.g. anti-aging; dermal regeneration for wrinkles and other skin defects; promotion of hair follicle growth, nerve and other organelle regeneration; adipose tissue regeneration; subdermal fat content; anti-bruising; topical ointment or patch with or without antibiotic(s); corneal repair; localized treatment for HIV or diabetic or age associated lipodystrophy; healing without scarring, or re-healing to diminish scarring; and wound bandage or dressing.

Advantages of the invention which is based on activating endogenous stem cells, avoids many of the difficulties associated with current stem cell therapeutic regimens: no stem cell isolation, no prep, no surgery or extraction; no in vitro propagation or re-transplantation; no immune response to transplanted cells or additives; no selection of the appropriate starter cell type; and no addition of cocktails of growth factors, feeder cells, other agents.

### Kits

The present invention provides articles of manufacture and kits containing materials useful for treating the pathological conditions described herein. The article of manufacture may include a container of a medicament as described herein with a label. Suitable containers include, for example, bottles, vials, syringes, and test tubes. The containers may be formed from a variety of materials such as glass or plastic. The container holds a composition having an active agent which is effective for treating, for example, diseases characterized by skin degeneration or damage. The label on the container indicates that the composition is used for treating skin diseases or injury and may also indicate directions for administration and monitoring techniques, such as those described above.

The kit of the invention includes the container described above and a second container, which may include a pharmaceutically acceptable diluent. It may further include other materials desirable from a commercial and user standpoint, including other buffers, diluents, filters, needles, syringes, and package inserts with instructions for use.

This invention also may be utilized for skin regeneration, including hypodermis, dermis, and epidermis. Similar procedures for skin filling have been used for hyaluronan.

### EXAMPLES

Without intent to limit the scope of the invention, exemplary instruments, apparatus, methods and their related results according to the embodiments of the present invention are given below. Note that titles or subtitles may be used in the examples for convenience of a reader, which in no way should limit the scope of the invention. Moreover, certain theories are proposed and disclosed herein; however, in no way they, whether they are right or wrong, should limit the scope of the invention so long as the invention is practiced according to the invention without regard for any particular theory or scheme of action.

### Example 1

### Regenerating Dermal and Subdermal Tissue in Humans

This example illustrates a composition and method of local application of the composition according to the invention, which is suitable for regenerating dermal and subdermal tissue in humans (and other mammals). The local treatment dose, which is at or near physiological levels, avoids side effects.

COMPOSITION A

| Component | Concentration for injection | |
|---|---|---|
| Hyaluronic Acid | 40 | mg/mL |
| IGF-1 | 200 | ng/mL |
| Saline for injection | to 1 | mL |

Components are combined under sterile conditions by mixing with the addition of surfactants and lowering the pH until nanoparticles are formed.
Hyaluronic acid injectable (e.g. Juvederm ® or Restylane ®)
IGF-1 injectable (e.g. Increlex ®)
ng = nanogram; mg = milligram; mL = milliliter

The formed Composition A is dispensed into multiple sterile syringes or vials for use. All materials are sterilized using known sterilization equipment and techniques, for example, autoclaves, convection ovens, or filtering with 0.2 micron filter.

Initially, 0.5 mL of Composition A is injected for localized delivery (alternatively, by micro-puncture, topical crème, patch, or other method of local delivery) into several locations in the dermis or hypodermis of the dorsal side of the hand (or alternatively injected into areas of the face, foot, dorsal hand or forearm, kneecap, or any other areas where the hypodermal layer is to be regenerated). In the other hand, placebo is injected with the same methods of injection. The methods of injection of Composition A include using as fine gauge needle as possible to reduce pain and injury, and injecting multiple small volumes in a pattern across the dorsal hand in order to distribute Composition A to areas requiring regeneration.

Regeneration of the subcutaneous (hypodermal) layer is measured in both hands with calipers before and after injection, and at 2, 4, and 6 weeks after injection. In some cases, a skin punch is obtained and subsequently prepared for histology by thin slicing and Hematoxylin/Eosin (H&E) staining.

### Example 2

### Regenerating Dermal and Subdermal Tissue in Mice

This example illustrates a composition and method of local application of the composition according to the invention, which is suitable for regenerating dermal and subdermal tissue in mice. The local delivery of Composition B into the mid to deep dermis, or hypodermis, creating a depot for sustained local release, regenerates subcutaneous tissue, evidenced by increased thickness of the hypodermal layer, an increased number of regenerated fat cells, sensory receptors, and hair follicles in mice (see figure).

Initially, each mouse is injected with 0.8 mL of Composition B, or appropriate controls, with a needle and syringe in 4 locations on the dorsal area (approximately 0.2 mL per location).

Each of 4 mice is injected in four dorsal sites per mouse with a needle and syringe for a total of 0.8 mL per mouse. In the composition, hyaluronic acid is administered at a concentration of 20 mg/mL and IGF-1 is administered at 500 ng/mL.

The following Composition B is prepared for injection into the mid to deep dermis, or hypodermis.

COMPOSITION B

| Component | Concentration for injection | |
|---|---|---|
| Hyaluronic Acid | 20 | mg/mL |
| IGF-1 | 500 | ng/mL |
| Saline for injection | to 1 | mL |

Hyaluronic acid and IGF-1 are obtained from R&D Systems (www.rndsystems.com).
ng = nanogram; mg = milligram; mL = milliliter

The composition is prepared by first creating a stock solution of IGF-1 by diluting IGF-1 to 0.1 mg/mL in saline for injection. Next, Composition B is prepared by combining 1.0 mL of a hyaluronic acid solution/gel with the 0.1 mg/mL IGF-1 stock solution. The components of Composition B are combined under sterile conditions by using 2 syringes with a sterile connector.

H&E histology slides from 6 mm mouse skin punches from animals treated with Composition B demonstrate increased thickness of the hypodermal layer (clear fat cell layer), an increased number of regenerated fat cells (clear), sensory receptors (blue/purple with red center), and hair follicles (blue/purple). (See FIG. 1C). Demonstrated in mice, this matrix formulation of hyaluronic acid and IGF-1, recruits and activates existing stem cells, regenerating skin, including hair follicles, sweat glands, sensory receptors (nerves), dermis and epidermis.

### REFERENCES

Wound healing composition of IGF-I and TGF-beta. Harry N. Antoniades et al U.S. Pat. No. 4,983,581.

Cosmetic composition Martin R. Green A composition suitable for topical application to mammalian skin or hair. U.S. Pat. No. 5,037,643

Price R D, Berry M G, Naysaria H A. Hyaluronic acid: the scientific and clinical evidence. J Plast Reconstr Aesthet Surg. 2007 Apr. 25

Carruthers J, Carruthers A. Hyaluronic acid gel in skin rejuvenation. J Drugs Dermatol. 2006 November-December; 5(10):959-64.

Hamilton T K. Assessing nonsurgical options for facial restoration. Dermatol Ther. 2007 March; 20 Suppl 1:S5-9. Review.

Cross-linked gels of hyaluronic acid and products containing such gels. Endre A. Balazs et al. U.S. Pat. No. 4,636,524

Locally produced IGF-I by condylar cartilages played an important role in the growth and remodeling of mandibular condyle after functional mandibular protrusion. Zhou Z. and Luo S. Abstract from the IADR/AADR/CADR 80th General Session (Mar. 6-9, 2002) (iadr.confex.com/iadr/2002SanDiego/techprogram/abstract_{—}9179.htm; accessed Aug. 16, 2010)

Circulating stem cells and uses related thereto. Blau, et al. US Patent Pub. No. 20060003312

Prior injury accelerates subsequent wound closure in a mouse model of regeneration. Davis T A et al. Cell Tissue Res. 2005 June; 320(3):417-26. Epub 2005 Apr. 23

Kim S J, Hahn S K, Kim M J, Kim D H, Lee Y P. Development of a novel sustained release formulation of recombinant human growth hormone using sodium hyaluronate microparticles. Kim S J et al., J Control Release. 2005 May 18; 104(2):323-35. Epub 2005 Apr. 7.

Method and composition for restoration of age related tissue loss in the face. Vincent C. Giampapa, US Patent Pub. No: US 2006/0073178 A1.

All publications and patent applications cited in this specification are incorporated herein by reference as if each individual publication or patent application were specifically and individually indicated to be incorporated by reference.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claim.

1. A pharmaceutical composition for repair or regeneration of dermal tissue, the composition comprising:
(a) a therapeutically effective amount of a growth factor for activating endogenous stem cells; and
(b) a bioabsorbable polymer.

2. The composition of claim 1, wherein the growth factor is insulin-like growth factor-1 (IGF-1).

3. The composition of claim 1, wherein the pharmaceutical composition is a sustained-release composition comprising an amount of the growth factor sufficient for activating endogenous stem cells for a period of time sufficient for repair and regeneration of dermal tissue.

4. The composition according to claim 1, wherein the bioabsorbable polymer is selected from the group consisting of hyaluronan, hydroxyapatite; polymers of glycolide, lactide and c-caprolactone; collagen; cross-linked hyaluronic acid; multiple different MWs HA; any suitable matrix forming agent; bioabsorbable gels; transplant-based fillers, fibroblasts or fat cells; synthetic Polymer-Based Fillers, e.g. polyacrylamide gel (Aquamid®), polyalkylimide gel (Bio-Alcamid®), Poly-L-lactic-acid (Sculptra/NewFill®), Polymethylmethacrylate (ArteFill/ArteColl®), elastin, microspheres, depleted natural or synthetic human skin matrix.

5. The composition of claim 4 wherein the bioabsorbable polymer is Lactel®.

6. The composition of claim 4 wherein the bioabsorbable polymer is recombinant human collagen.

7. The composition of claim 4 wherein the bioabsorbable polymer is hyaluronan.

8. The composition of claim 4 wherein the bioabsorbable polymer is depleted natural or synthetic human skin matrix.

9. The composition of claim 4 wherein the bioabsorbable polymer is poly L lactic acid.

10. The composition according to claim 1, wherein the composition comprises a nanoparticle.

11-16. (canceled)

17. The composition according to claim 2, wherein the IGF-1 is provided at a sustained-release dosage between 20 and 500 ng/mL.

18. The composition according to claim 2, wherein the IGF-1 is provided at a sustained-release dosage selected from the group consisting of 20, 50, 100, 200, 300, 200, or 500 ng/mL.

19. The composition according to claim 17, wherein the composition provides sustained release of less than 500, 400, 300, 200, 100, or 50 ng/mL of IGF-1 over 1, 2, 3, 4, 12, or 24 hours, respectively.

20. The composition according to claim 7, wherein the hyaluronan is present at a concentration between 0.02 mg/mL and 40 mg/mL.

21. The composition according to claim 1, wherein IGF-1 and hyaluronan (HA) are present at a IGF-1:HA ratio of: 1:40, 1:400, 1:4,000, 1:40,000, or 1:400,000, (w/w).

22. The composition claim 21, wherein IGF-1 and hyaluronan (HA) are provided in amounts sufficient to cause tissue regeneration when administered locally over 0.25 to 6 cm2 surface area or 2 to 1000 cm3 infused volume.

23. The composition of claims 22 wherein the IGF-1 is released at serum levels insufficient to cause side effects, adverse reactions or hypoglycemia.

24. The composition according to claim 17, formulated for therapeutical treatment for a disease or condition selected from the group consisting of: fat pad regeneration; hair regeneration; skin regeneration with nerves & organelles; wound healing, including diabetic ulcers; burn healing; skin regeneration and repair; enhance skin quality or appearance; prevention or remediation of skin disorders; diminishment or abolishment of scar tissues; breast skin regeneration (post surgery); cosmetic applications, anti-aging application, dermal regeneration for wrinkles and other skin defects; promotion of hair follicle growth, nerve and other organelle regeneration; adipose tissue regeneration; subdermal fat content; anti-bruising; topical ointment or patch with or without antibiotic(s); corneal repair; localized treatment for HIV or diabetic or age associated lipodystrophy; healing without scarring, or re-healing to diminish scarring; and wound bandage or dressing.

25. The composition of claim 20, wherein the treatment is prophylactic or therapeutic.

26. The composition according to claim 1, wherein a local application of the composition results in a sustained release of growth factors at the localized sites for activating endogenous stem cells for a period of time sufficient for repair and regeneration of dermal cells.

27. A method for repairing or regenerating skin cells, the method comprising:
(a) providing a pharmaceutical composition comprising a growth factor and a bioabsorbable polymer according to claim 1; and
(b) applying the composition to a site in need thereof for a period of time sufficient for repair and regeneration of the skin cells.

28. The method of claim 27, wherein the growth factor is IGF-1.

29. The method according to claim 27, wherein the bioabsorbable polymer is hyaluronan.

30. The method according to claim 27, wherein the composition is applied by a method selected from the group consisting of: topical application, injection, insertion of a stent, a dermal patch, and a microneedle array.

31. The method according to claim 27, wherein the composition is applied to an area comprising 0.25, 0.5, 0.75, 1.0, 2.0, 3.0, 4.0, 5.0, or 6.0 cm2.

32. The method of claim 27, wherein the composition is delivered to the hypodermis, dermis or epidermis.

33-38. (canceled)

39. The method according to claim 27, wherein the composition is applied in a manner sufficient to retain a reservoir of growth factor localized at the site for sustained release for a period of time sufficient for repair and regeneration of the skin cells.

40. The method of claim 39, wherein the composition comprising a single dose of IGF-1 selected from 20, 50, 100, 200, 300, 200 and 500 ng, combined with hyaluronan, is administered locally over a 0.25 to 6 cm2 surface area or a 2 to 1000 cm3 infused volume.

41. The method of claim 40, wherein administration of hyaluronan and IGF-1 result in reduced side effects or adverse reactions.

42. The method of claim 41, wherein the side effect or adverse reaction is hypoglycemia.

43. The method according to claim 27 wherein the repairing or regenerating skin cells is related to treatment of a disease or condition selected from the group consisting of: fat pad regeneration; hair regeneration; skin regeneration with nerves & organelles; wound healing, including diabetic ulcers; burn healing; skin regeneration and repair; enhance skin quality or appearance; prevention or remediation of skin disorders; diminishment or abolishment of scar tissues; breast skin regeneration (after surgery); cosmetic applications, e.g. anti-aging; dermal regeneration for wrinkles and other skin defects; promotion of hair follicle growth, nerve and other organelle regeneration; adipose tissue regeneration; subdermal fat content; anti-bruising; topical ointment or patch with or without antibiotic(s); corneal repair; localized treatment for HIV or diabetic or age associated lipodystrophy; healing without scarring, or re-healing to diminish scarring; and wound bandage or dressing.

44. The method of claim 43, wherein the treatment is prophylactic or therapeutic.

45. A method for preparing a nanoparticular pharmaceutical composition for repairing or regenerating skin cells, the method comprising:
(a) providing a therapeutically effective amount of a growth factor for activating endogenous stem cells;
(b) providing a bioabsorbable polymer; and
(c) combining the growth factor and the polymer under sterile conditions by mixing with the addition of surfactants and lowering the pH until nanoparticles are formed.

46. The method of claim 46 wherein the growth factor is IGF-1 and the polymer is hyaluronan.
